(19) United States
(12) Patent Application Publication
(10) Pub. No.: US 2016/0113981 A1
(43) Pub. Date: Apr. 28, 2016

(54) TOPICAL COMPOSITION

(71) Applicant: Angela Hooks Reaves, Northport, AL (US)

(72) Inventor: Angela Hooks Reaves, Northport, AL (US)

(21) Appl. No.: 14/923,169

(22) Filed: Oct. 26, 2015

Related U.S. Application Data
(60) Provisional application No. 62/068,318, filed on Oct. 24, 2014.

(51) Int. Cl. A61K 36/53; A61K 36/534; A61K 45/06; A61K 36/32; A61K 36/886; A61K 36/28; A61K 36/328

(52) U.S. Cl. CPC A61K 36/53; A61K 36/28; A61K 36/534; A61K 36/328; A61K 36/32; A61K 36/886; A61K 45/06

(57) ABSTRACT

A topical composition is provided that includes at least three plant extracts selected from the group consisting of: lavender, calendula, rosemary, peppermint, myrrh, frankincense, helichrysum and aloe. The plant extracts in are dissolved or suspended in a carrier. The topical composition in some embodiments has each of said at least three plant extracts present in an amount of from 0.1 to 60 total weight percent of an inventive composition, with the total amount of plant extracts being between 2 and 90 total weight percent. A method of treating irradiated skin includes the application of such a composition to skin after medical irradiation, then allowing sufficient time for the skin to heal. A method of inhibiting radiation dermatitis includes the application of such a composition to skin prior to medical irradiation, then irradiating the skin ionizing radiation.

### RELATED APPLICATION

This application claims priority benefit of U.S. Provisional Application Ser. No. 62/068,318 filed 24 Oct. 2014; the contents of which are hereby incorporated by reference.

### FIELD OF THE INVENTION

The present invention in general relates to a plant-based topical composition, and in particular, to a process for application of such a composition to skin suffering from radiation dermatitis or similar aggravated skin condition.

### BACKGROUND OF THE INVENTION

For treating wounds, the use of solutions of chemical products, such as Mercurochrome quaternary ammonium salts and synthetic antibiotics are well known. Although widely used, in particular in first interventions, these solutions exhibit the disadvantage of weakening the cell material in which the injury is located, and of being only partially effective.

Radiation dermatitis in most common form is sunburn, and also exhibited in patients undergoing radiation therapy and subjects exposed to nuclear radiation. On a cellular level, radiation dermatitis involves severe erythema with the cellular damage resulting in slow resolution and is often associated with cell apoptosis or even radiation-induced cancer due to the ability of ionizing radiation to interact with and damage DNA. Due the unique damage associated with high dose radiation experienced in medical treatments, conventional creams functioning as antibiotics, anti-inflammatories, burn salves, or combinations thereof have proven less than effective.

Owing to the discomfort, duration, and propensity of radiation dermatitis to lead to secondary complications, there exists a need for a composition to reduce the inflammation and pain associated with it, and help protect the skin from infection. There also exists a need for a composition when applied prior to radiation treatment, is able to delay the onset of radiation dermatitis based on a normalized dosimetry.

### SUMMARY OF THE INVENTION

A topical composition is provided that includes at least three plant extracts selected from the group consisting of: lavender, calendula, rosemary, peppermint, myrrh, frankincense, helichrysum and aloe. The plant extracts in are dissolved or suspended in a carrier. The topical composition in some embodiments has each of said at least three plant extracts present in an amount of from 0.1 to 60 total weight percent of an inventive composition, with the total amount of plant extracts being between 2 and 90 total weight percent.

A method of treating irradiated skin includes the application of such a composition to skin after medical irradiation, then allowing sufficient time for the skin to heal.

A method of inhibiting radiation dermatitis includes the application of such a composition to skin prior to medical irradiation, then irradiating the skin ionizing radiation.

### DETAILED DESCRIPTION OF THE PREFERRED EMBODIMENTS OF THE INVENTION

The present invention has utility as a topical composition. The inventive topical composition is well suited to treat radiation dermatitis, as well as other forms of dermatitis. When an inventive topical composition is applied prior to non-solar radiation exposure, the dosimetry onset of radiation dermatitis is delay or manifest as less severe.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

An inventive topical composition is form as a liquid or formed into an emulsion and contains at least three of the following plant extracts. In some embodiments 4, 5, 6, 7, and even all 8 of the following extracts are present in an inventive composition. These extracts include Lavender (Lavender Officinalus), Calendula (Calendula Officinalis), Rosemary (Rosmarinus Officinalis), Peppermint (Mentha Piperita), Frankincense (Boswellia Carteri), Myrrh (Commiphora Myrrha), Helichrysum (Helichrysum splendidum), Aloe (Aloe Vera), and the combination thereof.

The lavender extract contains linalool, perillyl alcohol, linalyl acetate, camphor, limonene, tannins, triterpenes, coumarins, cineole, and flavonoids. The skin quickly absorbs lavender oil. The constituents linalool and linalyl acetate are detectable in the blood five minutes after topical application, peak at 19 minutes, and largely disappear from the blood within 90 minutes. Without intending to be bound to a particular theory, rosemary extracts in the present invention lavender extract (oil) also has an analgesic effect and an anti-anxiety effect. Additionally, lavender extract is bactericidal with activity even against methicillin-resistant Staphylococcus aureus (MRSA) and vancomycin-resistant enterococci (VRE). Antifungal effects are also noted with activity being known for Candida albicans and Trichophyton rubrum, as did the constituent linalool. The constituent of lavender extract, caffeic acid also is functional as an antioxidant. Additionally, linalool induced relaxation in PET tomography. Components of lavender appear to have cytotoxic properties with limonene and POH having been shown to inhibit tumor growth in rats by blocking initiation and by promoting apoptosis.

Calendula extract contains oleanic acid, triterpenoid esters (betulinic acid, sterols, and triterpenoid saponins) carotenoids flavoxanthin, auroxanthin, methanol, carotenoids. At least eight bioactive triterpendiol monoesters have been identified in the extracts of dried calendula flowers: faradiol-3-O-palmitate, faradiol-3-O-myristate, faradiol-3-O-laurate, arnidiol-3-O-palmitate, arnidiol-3-O-myristate, arnidiol-3-O-laurate, calenduladiol-3-O-palmitate, and calenduladioI-3-O-myristate. The flavonoids, particularly patulitrin and patuletin, may be used as dyes. The saponin fractions isolated from Calendula officinalis flower include oleanolic acid (205.53 mg/g dry weight) and ursolic acid. Calenda saponins A, B, C, and D, two additional ionone glucosides (officinosides A and B), and two sesquiterpene oligoglycosides (officinosides C and D) have been isolated from the flowers of Egyptian Calendula officinalis.” Without intending to be bound to a particular theory, calendula extracts in the present invention confer anti-inflammatory, immunostimulating, antibacterial, antiviral, antiprotozoal, and antineoplastic properties. These specifically include inhibitory activity of 5-lipoxygenase (5-LO) and cyclooxygenase-2 (COX-2), key enzymes in inflammatory pathways. Antimicrobial properties have been observed for hydroacetonic extracted from fresh plants that inhibited the growth of Staphylococcus aureus at a concentration of 1 mg/mL in vitro. Oxygenated terpene alcohols and terpene lactones from calendula have been observed to possess trichomonacidal activity. Triterpenoid saponins from Calendula arvensis have inhibited multiplication of vesicular stomatitis virus and rhinovirus in vitro. Triterpene and flavonol glycosides isolated from calendula have also demonstrated inhibitory activity against Epstein-Barr virus activation. Wound healing is noted for these extracts through reduction of epithelialization time, an increase in wound strength, and improvement of wound contraction in rats with experimental incision wounds that were topically treated with calendula. Also, saponins isolated from calendula express in vitro antimutagenic and tumor cell cytotoxic activity. The active components of the anti-inflammatory activity of calendula are thought to be the triterpenoids, particularly faradiol monoester. Free ester faradiol is the most active and exhibits the same effects as an equimolar dose of indomethacin.

Rosemary extracts contain cuminic acid, bornyl acetate, caryophyllene, monoterpene hydrocarbons (alpha and beta-pinene), camphene, limonene, camphor, borneol, cineole, linalool, and verbinol. Rosemary contains a wide variety of volatile and aromatic components. Flavonoids in the extract include diosmetin, diosmin, genkwanin, luteolin, hispidulin, and apigenin. Phenols in rosemary extracts include caffeic, chlorogenic, labiatic, neochlorogenic, and rosmarinic acids, as well as salicylates. Without intending to be bound to a particular theory, rosemary extracts in the present invention confer antimicrobial, anti-cancer and anti-oxidative effects. Activity against certain bacteria including Staphylcoccus aureus, S. albus, Vibrio cholerae, Escherichia coli, Pseudomonas, Lactobacillus, and Corynebacteria has been observed, as well as efficacy against the fungi Candida albicans and Aspergillus parasiticus. Carnosol and carnosic acid are believed to account for more than 90% of the antioxidant properties of rosemary extract.

Peppermint extract contains among other constituents menthol (40.7 wt. % of the extract) and menthone (23.4%). menthyl acetate, 1,8-cineole, limonene, beta-pinene carvone, jasmone, carvacrol, limonene, phellandrene, and beta-caryophyllene. Menthol has a pain cessation cooling effect on skin. Peppermint extract has antibacterial properties against Escherichia coli, Helicobacter pylon, methicillin-sensitive and methicillin-resistant strains of Staphylococcus aureus, Pseudomonas species, Enterobacter aerogenes, and Salmonella enteritidis; as well as some antifungal and antiviral activity.

Frankincense contains sesquiterpene, camphene, dipentene, pinene, phellandrene, olibanol, boswellic acid (3-alpha-hydroxy-urs-12-en-23-oic acid), volatile oils, terpinols, arabinose, xylose, galactose, uronic acids, beta-sitosterin and phlobaphenes. Without intending to be bound to a particular theory, frankincense extracts in the present invention confer anti-inflammatory effects and include specific inhibitors of 5-lipoxygenase, the key enzyme of leukotriene biosynthesis.

Myrrh extract contains myrrholic acid, cinnamic acid, cuminic acid, eugenol, cadinene, pinene, dipentene, heerabolene, limonene, furanodiene-6-one and methoxyfuranoguaia-9-ene-8-one. Without intending to be bound to a particular theory, myrrh extracts in the present invention confer anti-tumoral effects and anti-microbial effects. Specifically, furanodiene-6-one and methoxyfuranoguaia-9-ene-8-one are active against Pseudomonas aeruginosa, Escherichia coli, and Candida albicans.

Helichrysum contains α-pinene, β-pinene, β-cadinene, Caryophyllene, 1,8-cineol. Without intending to be bound to a particular theory, helichrysum extracts in the present invention confer anti-inflammatory, vulnerary, cicatrisant, febrifuge, antimicrobial, anti-allergic effects.

Aloe (Aloe Vera) contains Arachidonic acid, y-linolenic acid, steroids (campestrol, cholesterol, (3-sitosterol), triglicerides, triterpenoid, glucomannan acemannan, aloeride, gibberillin, lignins, potassium sorbate, salicylic acid, bradykininase, maloyl glucan, veracylglucans, aloe-emodin, and uric acid. Without intending to be bound to a particular theory, aloe in the present invention confers anti-inflammatory, radioprotective effects. Also the polysaccharide glucomannan is an effective human skin moisturizer. Acemannan accelerates wound healing. The macrophage stimulating principle of acemannan appears to reside in the high molecular weight polysaccharide Aloeride. Acemannan has also been reported to exhibit antineoplastic and antiviral effects in vitro. Bradykininase, contributes anti-inflammatory properties and magnesium lactate, antipruritic effects. Salicylic acid and other antiprostaglandin compounds contribute local anti-inflammatory activity. Maloyl glucans also likely act as anti-inflammatories. Veracylglucans confer anti-proliferative effects. Aloe-emodin is thought to inhibit endothelial cell proliferation and inhibits tumor cell proliferation.

As used herein, a “plant extract” is a concentrated pharmaceutical preparations of plants obtained by removing the constituents active on human skin therefrom with a suitable solvent and evaporating all or nearly all the solvent, and adjusting the residual mass or powder to a prescribed standard. Solvents operative herein include water, C1-C20-alcohols, -ethers, -esters, -acids, -alkanes, and -alkenes; and forms thereof containing a substituent. Substituents illustratively including carbonyl-, carboxyl-, amino-, amido-, hydrodroxyl-, and sulfonyl-moities; and combinations thereof. It is appreciated that elevated temperature plant extractions such as steam-distilled hydrosols are intended to be encompassed herein.

Each of the at least three plant extracts is present in an amount of from 3 to 99 total weight percent of an inventive composition, with the total amount of plant extracts being between 1 and 97 total weight percent. It is appreciated that in addition to the at least three plant extracts, that other of the aforementioned extracts is readily provided in amounts of less than 1 total weight percent with the proviso that that total weight percent does not exceed 99 total weight percent. In alternate inventive embodiments, an individual plant extract is present from 1 to 10 total weight percent.

The preparation of the composition according to the invention includes bringing these constituent plant extracts together in determined and precise proportions and in determined and precise forms, and this causes a synergy in therapeutic effect pre-emptively, after, or both as to skin experiencing radiation dermatitis. The plant extracts provided in a carrier. In some embodiments, adjuvants are also intermixed with the carrier and plant extracts. The inventive topical composition obtained gives effects and results which are considerably improved in comparison with the effects and results obtained with each constituent taken in isolation and successively or in a cumulative manner, and this is unexpected and surprising based on the prior art.

The plant extracts of the present invention are dissolved or suspended in oil base according to certain inventive embodiments. Oils suitable for suspension or dissolution of plant extracts are limited only by storage stability with the plant extracts and skin compatibility. Oils operative herein illustratively include plant based oils such as olive oil, grapeseed oil, almond oil, jojaba oil, safflower oil, corn oil, peanut oil, sesame oil, cannola oil, soy oil, soybean oil, burdock root oil, tea tree oil, coconut oil, apricot seed oil, walnut oil, and combination thereof and animal based oils such as shark liver oil, cod liver oil and fish liver oil; and combinations thereof. In certain inventive embodiments, the carrier oil is selected to operate synergistically with the plant extracts by imparting therapeutic properties. By way of example apricot kernel oil has the attributes on human skin of being anti-inflammatory and hypo-allergenic, while coconut oil fractions have the attributes of stability towards essential oils in the inventive plant extracts, anti-fungal, antibacterial, and antioxidant effects. It is appreciated that an inventive composition is readily formulated with a carrier so as to be non-toxic and effectively edible as defined by an oral LD₅₀ of more than 1000 mg/kg.

Because the inventive topical compositions are applied to compromised skin in some inventive embodiments, all or some of the constituents of the composition are sterilized, either individually or together. In inventive embodiments, particularly where the composition may be applied to radiation burned skin that is broken, it is desired that at least some of the composition be sterile to help prevent infection at the treatment site. The sterilizing is readily performed, for example, by filter sterilization, for example by filtering through a 0.45 micron filter or through a 0.22 micron filter.

Any suitable sterilization method or combination of sterilization methods can be used, and can be used on any combination of composition and/or packaging components, as long as the sterilization does not adversely affect the therapeutic effectiveness of the inventive topical composition. For example, suitable sterilization techniques that may be employed include dry and moist heat sterilization, ionizing radiation (such as electron beam or gamma irradiation), exposure to gas, and aseptic filtration. Any suitable method or combination of methods can be used, and can be used on any combination of composition and/or packaging components, as long as the sterilization does not adversely affect the therapeutic effectiveness of the composition. It is appreciated that heat sterilization has a propensity to damage many plant extract components such as carbohydrates and proteins and as a result should be used with caution to avoid the loss of therapeutic effect.

An inventive composition may also contain at least one pharmaceutically-acceptable adjuvant illustratively including, waxes; paraffins; starch; tragacanth; a pluronic; cellulose and cellulose derivatives; silicones; bentonites; silicic acid; talc; zinc oxide; aluminum hydroxide; calcium silicates; alginate; acrylate; hyaluronic acid; polyethylene glycol; dimethyl sulfoxide (DMSO); vitamins such as E, ascorbic acid glucosides; partial glycerides of fatty acids; chitosan; and mixtures thereof.

In addition, the inventive topical composition may contain one or more compounds for burn treatment that are conventional to the art such as steroidal anti-inflammatories such as the prototypical cortisone; anti-bacterials such as silver sols, quinolones such as ciprofloxacin or levofloxacin; cephalosporins such as cefepime or ceflazidime; aminoglycoside such as gentamicin or amikacin; or Gram positive effective anti-biotics of amoxicillin, vancomycin, or linezolid; each alone or in combination. Additionally, cellular regrowth stimulating substance such as biotin, or epidermal growth factor are readily admixed with into an inventive topical composition.

In addition, the inventive topical composition may contain one or more compounds for improving cosmetic acceptability, including but not limited to, humectants, surfactants, fragrances, coloring agents, emollients, fillers, and the like. Furthermore, the inventive topical composition may be in the form of, for example, a paste, cream, gel, lotion, powder, spray, aerosol, or liquid. Where one or more of such other additives are present in the composition, and where the other components of the composition are sterilized as described above, it is preferred that the additional additives also be sterilized, so that sterility of the entire composition can be maintained. Such additives can be sterilized as described above.

The composition of the disclosure can be prepared in any suitable form, and the form can be tailored for the intended mode of application. For example, as desired, the composition can be made in the form of a serum, a cream, a lotion, a liquid, a spray, or the like. In embodiments, a cream or lotion form is desired, as it can be more precisely and easily applied to a treatment site, and can be made to stay in place on the treatment site for a sufficient time to provide a therapeutic effect.

It is appreciated that in addition to the treatment of human skin, an inventive topical composition is readily applied to therapeutic effect to animal skin that has been burned, infected, or irradiated. By way of examples such animals illustratively include dogs, cats, horses, cattle, sheep, primates, or rodents. Inventive compositions also show therapeutic effect on mucosal tissue and internal tissues of humans or animals.

An inventive topical composition may be applied prior to radiation treatment, immediately after burning the skin, such as within 1 to 120 minutes, after radiation exposure; shortly after burning the skin, such as within 2 to 4 hours; or some time thereafter such as 1-7 days after irradiation. Moreover, the composition may be applied to the burned skin a single time, or may be applied to the burned skin multiple times (e.g., at 30 minute intervals for about 5 hours).

An inventive topical composition may be applied so as to at least partially-cover the burned skin. In still other embodiments, the inventive composition can be applied so as to completely cover the burned skin. In still other embodiments, the inventive composition is applied so as to at least partially-cover unburned skin in the vicinity of the burned skin. For further protection, a covering is readily placed over the burned skin covered by the inventive composition. Any suitable covering may be used, and may be tailored to the location, type, and severity of the burn. For example, suitable coverings include bandages, wound dressings, gloves, and the like. In certain inventive embodiments, the covering may be placed over the burned skin after the composition has been applied to the burned skin.

To prepare an inventive composition, the plant extracts are prepared by conventional methods or purchased commercially. At ambient temperature, the plant extracts are added to a prepared carrier and additionally stirring performed as needed.

The present invention is further detailed with respect to the following non-limiting example.

### EXAMPLE

Plant extracts are mixed in an oil carrier as follows in the Table with the preservatives in:

| Constituent | Amount (total weight percent) |
|---|---|
| Actives: | 3-99% total |
| Lavender | 1-97% |
| Calendula | 1-97% |
| Rosemary | 1-97% |
| Peppermint | 1-97% |
| Myrrh | 1-97% |
| Frankincense | 1-97% |
| Helichrysum | 1-97% |
| Aloe | 1-97% |
| Carrier | remainder |
| Apricot Kernel Oil | |
| Coconut Oil Fractionated | |
| Rosemary Leaf Extract (preservative) | 0 to 3% |
| Vitamin E T50 (preservative) | 0 to 3% |

The resulting composition is applied to irradiated skin of subjects who receive radiation therapy. A comparison group applied conventional creams and ointments in dosimetry normalized subjects. With the both the inventive and conventional substances applied daily to cover the burned area, the inventive composition shows more rapid reduction in redness and subjects using the inventive composition reported less discomfort, including reduced swelling, redness, itching and irritation, and quicker return to “normal” appearance.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

1. A topical composition, comprising:
at least three plant extracts selected from the group consisting of: lavender, calendula, rosemary, peppermint, myrrh, frankincense, helichrysum and aloe; and
a carrier in which each of said at least three plant extracts are dissolved or suspend.

2. The topical composition of claim 1, wherein said at least three plant extracts are four plant extracts.

3. The topical composition of claim 1, wherein said at least three plant extracts are five plant extracts.

4. The topical composition of claim 1, wherein said at least three plant extracts are six plant extracts.

5. The topical composition of claim 1, wherein said at least three plant extracts are seven plant extracts.

6. The topical composition of claim 1, wherein said at least three plant extracts are eight plant extracts.

7. The topical composition of claim 1 wherein each of said at least three plant extracts is present in an amount of from 0.1 to 60 total weight percent of an inventive composition, with the total amount of plant extracts being between 2 and 90 total weight percent.

8. The topical composition of claim 1 wherein said carrier comprises a plant oil.

9. The topical composition of claim 8, wherein said plant oil is apricot kernel oil.

10. The topical composition of one of claims 8 further comprising coconut oil.

11. The topical composition of claim 1 further comprising an adjuvant.

12. The topical composition of claim 1 further comprising at least one of a steroidal anti-inflammatory, an anti-bacterial or a cellular regrowth stimulating substance.

13. The topical composition of claim 1 wherein said carrier is in the form of a serum.

14. The topical composition of claim 1 wherein said carrier is in the form of a cream.

15. The topical composition of claim 1 wherein said carrier is in the form of a spray.

16. The topical composition of claim 1 wherein one of said at least three plant extracts is at least 20%, with the remainder being adjuvants, carriers, and the like.

17. A method of treating irradiated skin comprising:
applying the composition of claim 1 to the skin after medical irradiation; and
allowing sufficient time for the skin to heal.

18. The method of claim 17 further comprising applying a bandage over the composition and the skin.

19. A method of inhibiting radiation dermatitis comprising:
applying the composition of claim 1 to the skin prior to medical irradiation; and
irradiating the skin with the medical radiation.
